(19) United States
(12) Patent Application Publication
(10) Pub. No.: US 2004/0043034 A1
(43) Pub. Date: Mar. 4, 2004

(54) Collectins as adjuvants

(76) Inventors: Jens Christian Jensenius, Odense M (DK); Anders Sjoholm, Lund (SE)

Correspondence Address: BROWDY AND NEIMARK, P.L.L.C., 624 NINTH STREET, NW, SUITE 300, WASHINGTON, DC, 20001-5303, US

(21) Appl. No.: 10/432,715

(22) PCT Filed: Nov. 27, 2001
(86) PCT No.: PCT/DK01/00786

(30) Foreign Application Priority Data
Nov. 27, 2000 (DK) PA 2000 01785

(51) Int. Cl. A61K 38/17; A61K 39/00

(52) U.S. Cl. 424/185.1; 514/8

(57) ABSTRACT

The present invention provides vaccine compositions comprising collectins e.g. mannose binding protein (MBL) and immunogenic determinants. Furthermore, the invention describes methods of immunising individuals with said compositions as well as the use of collectins for preparation of vaccine compositions.

### FIELD OF THE INVENTION

The present invention relates to vaccine compositions comprising collectins and immunogenic determinants. Furthermore, the invention relates to methods of immunising an individual with said compositions and to the use of collectins for preparation of vaccine compositions.

### BACKGROUND OF THE INVENTION

Animals have developed different complex strategies to protect themselves against infections. The immune responses can be divided into to main groups, the adaptive immune response, in which an adaptation has taken place and in which cells play a dominant part and the innate immune response, which is available instantly and which primarily is based on molecules present in the body fluids. The innate immune system is operational at time of birth, in contrast to the adaptive immune defence which only during infancy obtains its full power of protecting the body (Janeway et al., 1999).

Bacteria entering the body at mucosal surfaces or through broken skin are immediately recognised by collectins, a family of soluble proteins that recognise distinctive carbohydrate configurations that are present on the surfaces of microbes and absent from the cells of the multicellular organism. Collectins thus belong to the large and diverse group of pattern recognition receptors of the innate immune system. In humans, three collectins are known, although others may exist: cows for example have more. Collectins target the particles to which they bind either for uptake by phagocytes or for activation of the complement cascade, and in these ways can mediate their destruction.

Collectins all exhibit the following architecture: they have an N-terminal cysteine-rich region that appears to form inter-chain disulfide bonds, followed by a collagen-like region, an α-helical coiled-coil region and finally a C-type lectin domain which is the pattern-recognizing region and is referred to as the carbohydrate recognition domain (CRD). The name collectin is derived from the presence of both collagen and lectin domains. The α-helical coiled-coil region initiates trimerisation of the individual polypetides to form collagen triple coils, thereby generating collectin subunits each consisting of 3 individual polypeptides, whereas the N-terminal region mediates formation of oligomers of subunits. Different collectins exhibit distinctive higher order structures, typically either tetramers of subunits or hexamers of subunits. The grouping of large numbers of binding domains allows collectins to bind with high avidity to microbial cell walls, despite a relatively low intrinsic affinity of each individual CRD for carbohydrates.

C-type CRDs are found in proteins with a widespread occurrence, both in phylogenetic and functional perspective. The different CRDs of the different collectins enable them to recognise a range of distinct microbial surface components exposed on different microorganisms. The terminal CRDs are distributed in such a way that all three domain target surfaces that present binding sites has a spacing of approximately 53 Å (Sheriff et al., 1994; Weis & Drickamer, 1994). This property of ‘pattern recognition’ may contribute further to the selectively binding of microbial surfaces. The collagenous region or possibly the N-terminal tails of the collectins, are recognised by specific receptors on phagocytes, and is the binding site for associated proteases that are activated to initiate the complement cascade upon binding of the CRD domain to a target.

Mannan-binding lectin (MBL) also termed mannose-binding lectin or mannose binding protein is a collectin which has gained great interest as an important part of the innate immune system. MBL binds to specific carbohydrate structures found on the surface of a range of microorganisms including bacteria, yeast, parasitic protozoa and viruses, and has been found to exhibit antibacterial activity through killing mediated by activation of the terminal, lytic complement components or through promotion of phagocytosis. MBL deficiency is associated with susceptibility to frequent infections by a variety of microorganisms in childhood, and possibly also in adults.

The CRD of MBL recognises preferentially hexoses with equatorial 3- and 4-OH groups, such as mannose, glucose, N-acetylmannosamin and N-acetyl glucoseamin while carbohydrates which do not fulfil this sterical requirement, such as galactose and D-fucose, are not bound (Weis et al., 1992). The carbohydrate selectivity is obviously an important aspect of the self/non-self discrimination by MBL and is probably mediated by the difference in prevalence of mannose and N-acetyl glucoseamin residues on microbial surfaces, one example being the high content of mannose in the cell wall of yeasts such as Saccharomyces cerevisiae and Candida albicans. Carbohydrate structures in glycosylation of mammalian proteins are usually completed with sialic acid, which prevents binding of MBL to these oligomeric carbohydrates and thus prevents MBL recognition of ‘self’ surfaces. Also, the trimeric structure of each MBL subunit may be of importance for target recognition.

Complement is a group of proteins present in blood plasma and tissue fluid that aids the body's defences following an infection. The complement system is being activated through at least three distinct pathways, designated the classical pathway, the alternative pathway, and the MBLectin pathway (Janeway et al., 1999). The classical pathway is initiated when complement factor 1 (C1) recognises surface-bound immunoglobulin. The C1 complex is composed of two proteolytic enzymes, C1r and C1s, and a non-enzymatic part, C1q, which contains immunoglobulin-recognising domains. C1q and MBL shares structural features, both molecules having a bouquet-like appearance when visualised by electron microscopy. Also, like C1q, MBL is found in complex with two proteolytic enzymes, the mannan-binding lectin associated proteases (MASP). The three pathways all generate complement factor 3 (C3) convertase, which ensures the binding of C3b to the surface of the activating surface, i.e. the targeted microbial pathogen. Conversion of C3 into surface bound C3b is pivotal in the process of eliminating the microbial pathogen by phagocytosis or lysis (Janeway et al., 1999).

Certain O-antigen specific oligosaccharides of Salmonella have been reported to activate complement in C4-deficient guinea-pig serum and Salmonella serogroup C was later shown to react with MBL and hence activate complement by the MBLectin pathway, which is also termed the MBL pathway of complement activation or the lectin pathway.

It has for some time been speculated that the innate immune system may collaborate with the adaptive immune system in the generation of specific immune responses as exemplified by the antibody response after infection or vaccination. Fearon's group have shown that the attachment of the C3d fragment of complement factor C3 onto a protein antigen through fusion by gene technology can in crease the immunogenecity of the antigen 1000 fold or more. Practical applications of this technique, or any number of modifications, are still awaited.

The importance of the complement system for normal immune responses was first suggested by Pepys, who found impaired antibody responses to sheep erythrocytes, a thymus-dependent antigen, in mice that were C3-depleted with cobra venom factor. The idea of a link between innate and adaptive immunity was supported by reports demonstrating reduced primary antibody responses to thymus dependent antigens and impaired IgM to IgG switching in patients and experimental animals with deficiencies of C4, C2 and C3. The mechanism may involve the generation of C3-derived ligands for binding of antigen or antigen-containing complexes to complement receptors on B lymphocytes or antigen-presenting cells. Thus, blocking of CR1 (CD35) and CR2 (CD21) in mice with specific anti CR1 and anti-CR2 antibodies or with soluble receptor protein reduced antibody responses to immunisation and experiments with CR1 and CR2-deficient knock-out mice show the requirement of these receptors for responses to thymus-dependent antigens. In addition, patients with leucocyte adhesion deficiency, who lack the CD11/CD18 adhesion molecule CR3, demonstrate impaired antibody responses and failure to switch from IgM to IgG. The C3-derived fragment C3d, a specific CR2 ligand, as mentioned above, show a strong dose-dependent adjuvant effect.

Deficiencies of the classical complement pathway (C1, C2, C4 and C3) are associated with infections by encapsulated bacteria. The main reason for this is probably the reduced efficiency of opsonic and bactericidal defence mechanisms caused by complement dysfunction. However, impaired immune responses to polysaccharide antigens might also be considered. The influence of complement on responses to thymus-independent antigens has not been extensively studied, and the available information is contradictory. Thus, low antibody responses to thymus-independent antigens have been clearly documented in C3-depleted mice and C3-deficient dogs. On the other hand, some reports find that C3-deficient patients appear to respond normally to immunisation with polysaccharide vaccines.

Selander et al., 1999, describes that sera from patients with C2 deficiency has a marked reduction of IgG antibody to the Salmonella serogroup C oligosaccharide. Similarly, IgG antibody to the Salmonella serogroup C antigen was reduced in serum from individuals with very low MBL concentrations. However, IgM levels against the CO-antigen appeared normal in both groups. A possible explanation for these findings is that the lectin pathway, in conjunction with intact C4, C2 and C3 function, promotes IgG responses to MBL-binding carbohydrate structures in vivo by efficient production of C3d-coated antigen. Fearon & Locksley have suggested the possibility of such a course of events with focus on the primary immune response. One may also speculate on a more direct influence of MBL-carbohydrate complexes through interaction with MBL receptors on immunocompetent cells. MBL deficiency is a fairly common form of immunodeficiency associated with susceptibility to infection. An interesting implication is that interference with immune responses might be an integral part of this immunodeficiency syndrome.

Lutz recently discussed the basis of primary immune responses to nonself, and suggested that information of complement-activating imune complexes containing antigen and natural antibody may play a critical role in vivo. This concept could be valid for thymus-dependent as well as for some thymus-independent antigens. MBL might play a role parallel to that of natural antibody.

### SUMMARY OF THE INVENTION

The question emerges if reduced immune responses, secondary to complement dysfunction, contributes to the decreased immunity encountered in conditions associated with impaired recruitment of C3. An important point is that immune responses to thymus-independent antigens mainly appears to require complement when antigen is presented at low concentrations.

Maturation of carbohydrate immunity develops slowly during ontogeny and might partly be determined by a course of subclinical immunisation events with exposure to low doses of antigen. In this way, it is conceivable that complement deficiency might delay establishment of immunity. On the other hand, distinct vaccination responses to polysaccharides have been documented in deficiencies of C3and properdin, a component of the alternative activation pathway, and might also be expected in classical pathway deficiency states.

The findings of Selander et al, 1999 indicates an impaired immune responses against some carbohydrate antigens in C2 deficiency and suggests that the MBL pathway could be critically involved in isotype switching during immune system maturation.

The impact of complement dysfunction on immune responsiveness appears to reflect interesting links between innate and adaptive immunity and has clinical significance with regard to establishment of immunity in inherited and adaptive complement deficiency states.

Considering the importance of a proper, protective responses on 100% of vaccinations, underlines the significance of exploring avenues of enhancing the immunogenecity of vaccines, specially those which are known to have an unsatisfactory success rate. As an example, hepatitis B virus is causing not only hepatitis, but also trigers the development of liver cancer, the cause of more that 7000,000 deaths annually. Vaccination with hepatitis B surface antigen produces effective protection in 90%, but the remaining 10% fail to produce an antibody response on repeated vaccination.

It is therefore of great importance to provide improved vaccines with enhanced immunogenecity. Adjuvants are generally used to optimise the efficacy of an immunogenic composition. Adjuvants generally consist of agents that are included in the formulation used to provide and/or enhance the ability of the immunogenic composition to induce a desired immune response.

Accordingly, it is a first objective of the present invention to provide a composition comprising an pharmaceutically effective amount of at least one collectin and/or collectin homologue as an adjuvant and at least one immunogenic determinant for use as a vaccine.

It is a second objective of the present invention to provide a method for immunising an individual against an immunogenic determinant, comprising administering to said individual at least one collectin and/or collectin homologue and at least one immunogenic determinant selected from the group consisting of bacterial, fungal, viral and other pathogenic immunogenic determinants and any derivative of such infectious agents.

It is a third objective of the present invention to use a pharmaceutical acceptable amount of at least one collectin and/or collectin homologue for the preparation of a pharmaceutical composition for improvement of the immune response following vaccination with bacterial, fungal, viral and/or other pathogenic immunogenic determinants and/or any derivative of such infectious agents.

Furthermore it is an objective of the present invention to provide uses of a pharmaceutical acceptable amount of at least one collectin and/or collectin homologue and at least one immunogenic determinant selected from the group consisting of bacterial, fungal, viral and other pathogenic immunogenic determinants and any derivative of such infectious agents for the preparation of a vaccine composition.

It is a further objective of the present invention to provide a kit of parts comprising a pharmaceutically effective amount of a collectin and/or a collectin homologue as an adjuvant and at least one immunogenic determinant for use as a vaccine.

### DEFINITIONS

Adjuvant: Any substance whose admixture with an administered immunogenic determinant increases or otherwise modifies the immune response to said determinant.

Carrier: A scaffold structure, for example a polypeptide or a polysaccharide, to which an immunogenic determinant is capable of being associated.

Collectins: A family of structurally related, carbohydrate-recognising proteins of innate immunity, including mannan-binding lectin and surfactant proteins A and D. The name refers to the presence of a collagen-like region and a C-type lectin domain.

Complement: A group of proteins present in blood plasma and tissue fluid that aids the body's defences following an infection. Complement is involved in destroying foreign cells and attracting phagocytes to the area of conflict in the body.

Conjugated: An association formed between two compounds for example between an immunogenic determinant and a collectin and/or collectin homologue or between an immunogenic determinant and a saccharide. The association may be a physical association generated e.g. by the formation of a chemical bond, such as e.g. a covalent bond.

CRD: Carbohydrate recognition domain, a C-type lectin domain that is found at the C-terminus of collectins.

Immunogenic determinant: A molecule, or a part thereof, containing one or more epitopes that will stimulate the immune system of a host organism to make a secretory, humoral and/or cellular antigen-specific response, or a DNA molecule which is capable of producing such an immunogen in a vertebrate.

Immune response: Response to a immunogenic composition comprising an immunogenic determinant. An immune response involves the development in the host of a cellular- and/or humoral immune response to the administered composition or vaccine in question. An immune response generally involves the action of one or more of i) the antibodies raised, ii) B cells, iii) helper T cells, iv) suppressor T cells, v) cytotoxic T cells and iv) complement directed specifically or unspecifically to an immunogenic determinant present in an administered immunogenic composition.

MBL: Mannan-binding lectin

Vaccination: Process of inducing a protective immune response in an organism.

Vaccine: Immunogenic composition capable of raising a protective immune response in a subject.

### DESCRIPTION OF FIGURES

FIG. 1 illustrates the assignment of Salmonella O antigen-specific IgG antibody concentrations to operational reference sera (a-BO, a-CO, a-DO). The amount of immunofixed IgG at defined concentrations was assessed using dilutions of a human serum protein calibrator (X 0908) and wells coated with anti-Fab antibodies on one part of an ELISA microtiter plate. On the other part of the plate, dilutions of anti-BO, anti-CO and anti-DO sera were added to wells coated with Salmonella O antigen-specific oligosaccharides. Parallel curves permitted expression of the antibodies in μg/ml. Similar results were obtained with IgM and IgA antibodies in the assay system. The X 0908 curve for IgG was based on 10 experiments giving a 13.0% coefficient of variation for points in the linear part of the curve. Specific IgG measurements were repeated twice (10.7% coefficient of variation).

FIG. 2 illustrates IgG antibodies to Salmonella serogroups B, C and D (BO, CO and DO) in C2-deficient adults (C2D, closed symbols) and in normal human control sera (NHS, open symbols). Statistical analysis was performed with the two-tailed Mann-Whitney test (n.s.=not significant).

FIG. 3 illustrates IgM antibodies to Salmonella serogroups B, C and D. Abbreviations and symbols as given in FIG. 2.

FIG. 4 illustrates MBL levels in relationship to IgG antibodies to Salmonella serogroup C as determined in the normal human controls (top), C2-deficient adults (middle), and in healthy pesons with low MBL levels (bottom). The MBL measurements were performed by ELISA.

### DETAILED DESCRIPTION OF THE INVENTION

### Collectins

In one embodiment of the present invention a composition is provided that comprise at least one collectin and/or collectin homologue. Such collectin and/or collectin homologue preferably comprise one or more collectin subunits, such as for example 2 subunits (dimer) or for example 3 subunits (trimer) or for example 4 subunits (tetramer) or for example 5 subunits (pentamer) or for example 6 subunits (hexamer). More preferably, such collectin and/or collectin homologue comprise a mixture of collectins and/or collectin homologues, that each comprise a different number of subunits.

Preferably the ratio of tetramers, pentamers and/or hexamers to dimers of collectin subunits is at least 2:1, more preferably at least 3:1, yet more preferably at least 4:1, most preferably at least 5:1.

In one embodiment all subunits of said collectin and/or collectin homologue are alike.

Each collectin subunit preferably comprises 3 collectin polypeptides, that in a preferred embodiment are all alike. Such a collectin polypeptide chain, preferably comprises an N-terminal region cysteine rich region, a collagen-like domain, a neck region and/or a carbohydrate recognition domain (CRD), more preferably a collectin polypeptide chain comprises an N-terminal region cysteine rich region, a collagen-like domain, a neck region and a carbohydrate recognition domain (CRD).

Said carbohydrate recognition domain preferably is a C-type lectin domain, which preferably is capable of interacting with carbohydrates in a Ca²⁺ dependent manner.

Said neck region preferably comprise α helical secondary structure, that more preferably is capable of interacting with α-helices of two other neck regions to form a coiled coil type triple helix.

Said collagen-like domain preferably comprise repeats of the collagenous motif Gly-X-Y. Preferably, the Y position contains hydroxy prolines. In one embodiment the collagen-like domain comprise at least 1, such as at least 2, for example at least 5, such as at least 10, for example at least 15 repeats of the collagenous motif. Such repeat sequences may follow one another uninterruptedly or they may be interrupted. In one preferred embodiment they are interrupted at least once by one or more Gln residues.

In one embodiment of the present invention collectins are selected from the group consisting of: SP-A, SP-D, CL43, conglutinin, CL1 and mannan-binding lectin (MBL).

It has not escaped our attention that collectins might excert adjuvant activity through mechanisms not necessarily involving other complement factors. This is immaterial to this application which claims the adjuvant activity of collectins regardless of mechanism.

In one embodiment the composition of the present invention comprise additional complement factors. Such complement factors could for example be C2, C3, C4 or C5.

Collectins according to the present invention may furthermore be hybrids of collectins, for example hybrids of collectins selected from the group consisting of SP-A, SP-D, CL43, conglutinin, CL1, mannan-binding lectin (MBL) and homologues thereof. Accordingly, a collectin according to the invention may comprise one or more domains from one collectin and one or more other domains from a different collectin. The use of hybrids of collectins increases the spectrum of carbohydrates recognised by the different kind of collectins.

For example the N-terminal cysteine rich region may be derived from either SP-A, SP-D, CL43, conglutinin, CL1, MBL or homologues thereof and the collagen-like domain may be derived from either SP-A, SP-D, CL43, conglutinin, CL1, MBL or homologues thereof and the neck region may be derived from either SP-A, SP-D, CL43, conglutinin, CL1, MBL or homologues thereof and the carbohydrate recognition domain (CRD) may be derived from either SP-A, SP-D, CL43, conglutinin, CL1, MBL or homologues thereof.

In one example the N-terminal cysteine rich region, the collagen-like domain and the neck region may be derived from MBL, whereas the carbohydrate recognition domain (CRD) may be derived from either SP-A or SP-D. The resulting hybrids will be MBL like molecules recognising the same carbohydrates as SP-A or SP-D.

Furthermore, collectins according to the present invention may be hybrids between a collectin and an antibody. Such a hybrid may comprise one or more domains from one or more collectins. Preferably the hybrid comprises an N-terminal cysteine rich region, a collagen-like domain and/or a neck region, more preferably the hybrid comprises an N-terminal cysteine rich region, a collagen-like domain and a neck region. Furthermore, the hybrid may comprise one or more domains or fragments from an antibody. Preferably the hybrid may comprise an antigen binding fragment of an antibody for example an Fc fragment.

Accordingly, a collectin according to the present invention may in one example comprise the N-terminal cysteine rich region, the collagen-like domain and the neck region derived from MBL coupled to an antigen binding fragment of an antibody.

Preferably, the antigen binding fragment of the antibody is selected so that it may specifically interact with an antigen, which is comprised within the immunogenic determinant of the specific embodiment of the invention.

### MBL

MBL is one non-limiting example of a collectin, which may be employed with the present invention.

In one embodiment, MBL may be directly associated with the antigen. For example MBL may be naturally present on antigen or MBL may naturally be able to associate with the antigen. Alternatively, MBL may be chemically coupled to the antigen or it may be coupled to the antigen by any other useful method.

Preferably said MBL is mammalian MBL, more preferably human MBL. In particular MBL may be MBL of the sequence according to SEQ ID: 1 of international patent application 00/70043, said sequence corresponding to the MBL sequence having database accession NO: P11226.

Said MBL may be derived from any source known to the person skilled in the art. In one embodiment of the present invention the MBL is naturally occurring MBL. Preferably, such natural occuring MBL has been purified from mammalian plasma, more preferably from human plasma.

In a preferred embodiment the MBL is recombinant and expressed from a gene expression construct comprising nucleotide sequences encoding MBL polypeptides or functional homologues thereof operably linked to expression signals not natively associated therewith. Said nucleotide sequences encoding MBL polypeptides could for example be cDNA sequences or they could be genomic DNA sequences.

Said recombinant MBL could be produced in any suitable host known to the person skilled in the art, for example such host could be selected from the group consisting of transgenic animals, mammalian cell lines, which includes human cell lines, insect cells, yeast cells, bacterial cells and plants.

Preferably such recombinant MBL comprises oligomers of MBL subunits, more preferably recombinant MBL comprises oligomers of MBL larger than dimers. For example said recombinant MBL may comprise oligomers of MBL subunits, so that the ratio of tetramers, pentamers and/or hexamers to dimers of MBL subunits is at least 2:1, preferably at least 3:1, more preferably at least 4:1, most preferably at least 5:1.

In a preferred embodiment of the present invention at least 50% of the MBL oligomers has an apparent molecular weight higher than 200 kDa, when analysed by SDS-PAGE and/or Western blot. For example at least 60%, such as at least 70%, for example at least 80%, such as at least 90%, for example at least 95% of the MBL oligomers has an apparent molecular weight higher than 200 kDa, when analysed by SDS-PAGE and/or Western blot. In determination of the amount of MBL having an apparent molecular weight higher than 200 kDa densitometic analysis may be used wherein protein bands on a SDS-PAGE gel is stained with protein-staining, e.g. silver staining or Coomassie Blue staining, or specific staining of Western blot using an MBL specific antibody.

In one especially preferred embodiment recombinant MBL is produced and purified as described in patent application PCT/DK00/00246, which is hereby incorporated by reference in its entirety.

MASPs are serine proteases, which in complex with MBL, are involved in the MBL pathway of complement activation. In one embodiment of the present invention the composition furthermore comprise one or more serine proteases, preferably one or more MASPs, more preferably one or more activated MASPs. Preferably, said MASP is in a complex with the MBL or MBL homologue of the present invention. Said MASPs could be selected from the group consisting of MASP-1, MASP-2 and MASP-3, preferably said MASP is the activated form of MASP-2.

### Homoloques of Collectins

A homologue of a collectin including MBL within the scope of the present invention should be understood as any protein capable of exerting a function similar to the function of a collectin. In particular such function is the ability to activate complement upon binding to one or more saccharides.

A homologue may comprise one or more conservative amino acid substitutions, such as at least 2 conservative amino acid substitutions, for example at least 3 conservative amino acid substitutions, such as at least 5 conservative amino acid substitutions, for example at least 10 conservative amino acid substitutions, such as at least 20 conservative amino acid substitutions, for example at least 50 conservative amino acid substitutions such as at least 75 conservative amino acid substitutions, for example at least 100 conservative amino acid substitutions. Conservative amino acid substitutions within the meaning of the present invention is substitution of one amino acid within a predetermined group of amino acids for another amino acid within the same predetermined group, exhibiting similar or substantially similar characteristics. Such predetermined groups are for example:

i) polar side chains (Asp, Glu, Lys, Arg, His, Asn, Gln, Ser, Thr, Tyr, and Cys,)

ii) non-polar side chains (Gly, Ala, Val, Leu, Ile, Phe, Trp, Pro, and Met)

iii) aliphatic side chains (Gly, Ala Val, Leu, Ile)

iv) cyclic side chains (Phe, Tyr, Trp, His, Pro)

v) aromatic side chains (Phe, Tyr, Trp)

vi) acidic side chains (Asp, Glu)

vii) basic side chains (Lys, Arg, His)

viii) amide side chains (Asn, Gln)

ix) hydroxy side chains (Ser, Thr)

x) sulphor-containing side chains (Cys, Met), and

xi) amino acids being monoamino-dicarboxylic acids or monoamino-monocarboxylic-monoamidocarboxylic acids (Asp, Glu, Asn, Gln).

Conservative substitutions may be introduced in any position of a preferred collectin. It may however also be desirable to introduce non-conservative substitutions. A non-conservative substitution should lead to the formation of a homologue of a collectin capable of exerting a function similar to the function of said collectin. Such substitution could for example i) differ substantially in hydrophobicity, for example a hydrophobic residue (Val, Ile, Leu, Phe or Met) substituted for a hydrophilic residue such as Arg, Lys, Trp or Asn, or a hydrophilic residue such as Thr, Ser, His, Gln, Asn, Lys, Asp, Glu or Trp substituted for a hydrophobic residue; and/or ii) differ substantially in its effect on polypeptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

In a further embodiment the present invention relates to homologues of a preferred collectin, wherein such homologues comprise substituted amino acids having hydrophilic or hydropathic indices that are within +/−2.5, for example within +/−2.3, such as within +/−2.1, for example within +/−2.0, such as within +/−1.8, for example within +/−1.6, such as within +/−1.5, for example within +/−1.4, such as within +/−1.3 for example within +/−1.2, such as within +/−1.1, for example within +/−1.0, such as within +/−0.9, for example within +/−0.8, such as within +/−0.7, for example within +/−0.6, such as within +/−0.5, for example within +/−0.4, such as within +/−0.3, for example within +/−0.25, such as within +/−0.2 of the value of the amino acid it has substituted.

The importance of the hydrophilic and hydropathic amino acid indices in conferring interactive biologic function on a protein is well understood in the art (Kyte & Doolittle, 1982 and Hopp, U.S. Pat. No. 4,554,101, each incorporated herein by reference).

The amino acid hydropathic index values as used herein are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5) (Kyte & Doolittle, 1982).

The amino acid hydrophilicity values are: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4) (U.S. Pat. No. 4,554,101).

Substitution of amino acids can therefore in one embodiment be made based upon their hydrophobicity and hydrophilicity values and the relative similarity of the amino acid side-chain substituents, including charge, size, and the like. Exemplary amino acid substitutions which take various of the foregoing characteristics into consideration are well known to those of skill in the art and include: arginine and lysine; glutamate and aspartate; serine and threonine; glutamine and asparagine; and valine, leucine and isoleucine.

Furthermore, a homologue may comprise addition or deletion of an amino acid, for example an addition or deletion of from 2 to 100 amino acids, such as from 2 to 50 amino acids, for example from 2 to 20 amino acids, such as from 2 to 10 amino acids, for example from 2 to 5 amino acids, such as from 2 to 3 amino acids. However, additions of more than 100 amino acids, such as additions from 100 to 500 amino acids, are also comprised within the present invention.

Proteins sharing at least some homology with a preferred collectin are to be considered as falling within the scope of the present invention when they are at least about 40 percent homologous with the preferred collectin, such as at least about 50 percent homologous, for example at least about 60 percent homologous, such as at least about 70 percent homologous, for example at least about 75 percent homologous, such as at least about 80 percent homologous, for example at least about 85 percent homologous, such as at least about 90 percent homologous, for example at least 92 percent homologous, such as at least 94 percent homologous, for example at least 95 percent homologous, such as at least 96 percent homologous, for example at least 97 percent homologous, such as at least 98 percent homologous, for example at least 99 percent homologous with the preferred collectin.

### Immunogenic Determinant

The immunogenic determinant of the present invention could be any immunogenic determinant, against which it is desirable to raise a secretory, humoral and/or cellular antigen-specific immune response in the host organism. In particular, such immungenic determinant could be a bacterial, fungal, viral and/or other pathogenic immunogenic determinants and/or any derivative of such infectious agents.

In one preferred embodiment the immunogenic determinant comprise one or more saccharides. Said saccharide can be selected from the group consisting of mono-saccharides, di-saccharides, tri-saccharides, oligo-saccharides and poly-saccharides. Said di-saccharides, tri-saccharides and poly-saccharides may comprise mono-saccharide units only of one kind or they may comprise mono-saccharide units of more than one kind.

Each mono-saccharide unit can be selected from the group consisting of ketoses and aldoses. Ketoses are for example dihydroxyacetone (DHA), erythrulose, ribulose, xylulose, fructose and sorbose, as well as derivatives thereof. Aldoses are for example erythrose, threose, xylose, arabinose, ribose, deoxyribose, glucose, mannan (or mannose) and galactose, as well as derivatives thereof. Disaccharides can furthermore be selected from the group consisting of sucrose, lactose, maltose, cellobiose, trehalose and melibiose. Saccharides furthermore include mannitol, sorbitol, inositol, glucoheptose and L-lyxose.

Preferably, the saccharide is capable of interacting with the collectins of the present invention. When the collectin of the present invention is selected from the group consisting of MBL and MBL homologues the immunogenic determinant preferably comprises one or more mono-, oligo- or polysaccharides reactive with MBL, including for example mannan and mannose.

In one embodiment of the present invention the immunogenic determinant comprise saccharides as described above, which are not natively associated with said immunogenic determinant. Preferably, said saccharides are able to interact with the collectin of the present invention. In case the collectin is selected from the group consisting of MBL and MBL homologues, said saccharides, which for example may be mono-, oligo- or polysaccharides, preferably are reactive with MBL, including for example mannan and mannose.

Said saccharides is preferably conjugated to said immunogenic determinant by a covalent bond. The saccharide may be conjugated to the immunogenic determinant by any method known to the person skilled in the art. For example the method may be any of the methods described in Glycoconjugates, edited by Allen and Kisailus, Marcel Dekker Inc., in particular the methods described in chapter 6, p. 121 to 165. In addition the carbohydrate may be linked to the immunogenic determinant using any useful cross-linking agent available to the person skilled in the art. In particular bifunctional cross-linking reagents may be used, for example any of the reagents described in the 2001-2001 catalogue of Perbio, p. 294-343.

In another embodiment the immunogenic determinant is conjugated directly to the collectin of the present invention. Preferably said immunogenic determinant is conjugated to the collectin by a covalent bond. The immunogenic determinant may be conjugated to the collectin by any of the methods described herein above for conjugating saccharide to the immunogenic determinant. In a preferred embodiment, said immunogenic determinant is conjugated to MBL.

However, it is also comprised within the present invention that the immunogenic determinant and the collectin of the present invention may not associated directly with each other.

In one preferred embodiment the immunogenic determinant is one or more bacterial antigens and/or a derivatives of a bacterial antigens. Bacterial antigens could be derived from bacteria selected from the group consisting of: Bordetella pertussis, Brucella abortis, Escherichia coli, Salmonella species, salmonella typhi, Streptococci, Vibrio (V. cholera, V. parahaemolyticus), Shigella, Pseudomonas, Brucella species, Mycobacteria species (tuberculosis, avium, BCG, leprosy,), Pneumococci, Staphlylococci, Enterobacter species, Rochalimaia henselae, Pasterurella (P. haemolytica, P. multocida), Chlamydia (C. trachomatis, C. psittaci, Lymphogranuloma venereum), Syphilis (Treponema pallidum), Haemophilus species, Mycoplasmosis, Lyme disease (Borrelia burgdorferi), Legionnaires' disease, Botulism (Colstridium botulinum), Corynebacterium diphtheriae, Yersinia entercolitica and Rickettsiae.

Preferably such bacterial antigens are Salmonella, Steptococcus pneunomiae or meningococcus antigens, more preferably the immunogenic determinant is a Salmonella O-antigen.

In one embodiment of the present invention the immunogenic determinant is one or more viral antigens and/or a derivatives of viral antigens. Viral antigens could be derived from a virus selected from the group consisting of: Adeno-associated virsu, Adenovirus, Avian infectious bronchitis virus, Baculovirus, Chicken pox, Corona virus, Cytomegalovirus, Enterovirus, Epstein Barr virus, Feline leukemia virus, Foot and mouth disease virus, Hepatitis A, Hepatitis B, Hepatitis C, Hepatitis E, Herpes species, Herpes simplex, Influenza virus, HIV-1, HIV-2, HTLV 1, Influenza A and B, Kunjin virus, Lassa fever virus, LCMV (lymphocytic choriomeningitis virus), lentivirus, Measles, Mengo virus, Papilloma virus, Parovirus, Parainfluenza virus, Paramyxovirus, Parvovirus, Poko virus, Polio virus, Polyoma tumor virus, pseudorabies, Rabies virus, Reovirus, Respiratory syncytial virus, retrovirus, rhinovirus, Rinderpest, Rotavirus, Semliki forest virus, Sendai virus, Simian Virus 40, Sindbis virus, SV5, Tick borne encephalitis virus, Togavirus (rubella, yellow fever, dengue fever), Vaccinia virus, Venezuelan equine encephalomyelitis and Vesicular stomatis virus.

In one embodiment of the present invention such viral antigens are preferably influenza virus antigens or hepatitis B virus antigens.

The immunogenic determinant could further more be an antigen from a parasite selected from the group consisting of Malaria (Plasmodium. falciparum, P. vivax, P. malariae), Schistosomes, Trypanosomes, Leishmania, Filarial nematodes, Trichomoniasis, Sarcosporidiasis, Taenia (T. saginata, T. solium), Leishmania, Toxoplasma gondii, Trichinelosis (Trichinella spiralis) or Coccidiosis (Elmeria species).

The immunogenic determinant could further more be an antigen from a fungus selected from the group consisting of Cryptococcus neoformans, Candida albicans, Apergillus fumigatus and Coccidioidomycosis.

The specific immunogenic determinant can for example be a protein, a polysaccharide, a lipopolysaccharide or a lipopeptide; or it can be a combination of any of these. Particularly, the specific immunogenic determinant can include a native protein or protein fragment, or a synthetic protein or protein fragment or peptide; it can include glycoprotein, glycopeptide, lipoprotein, lipopeptide, nucleoprotein, nucleopeptide; it can include a peptide-peptide conjugate; it can include a recombinant nucleic acid expression product. Said proteins and/or peptides may be expressed in bacteria, yeast or mammalian cells, or alternatively may be isolated from viral preparations. Furthermore, peptides can be synthesised in vitro.

In one embodiment the immunogenic determinant is a live attenuated i.e., non-virulent, pathogen that have been “crippled” by means of genetic mutations. The mutations prevent the pathogens from causing disease in the recipient or subject to be vaccinated.

In another embodiment the immunogenic determinant is non-viable whole organisms. In particular, non-viable whole organisms are pathogens which have been inactivated either by chemical treatment, i.e., formalin inactivation, or by treatment with lethal doses of radiation.

In yet another embodiment the immunogenic determinant comprise a subcellular component purified from a pathogen against which it is desirable to immunise an individual. Such immunogenic determinant are usually safe to administer since it is unlikely that the subcellular components will cause disease in the recipient. The purified subcellular component may be either a defined subcellular fraction, purified protein, nucleic acid or polysaccharide having an immunogenic determinant capable of stimulating an immune response against the pathogen. The immunogenic determinants can be purified from a preparation of disrupted pathogens. Alternatively, the immunogenic determinants such as proteins, nucleic acids or polysaccharides may be synthesised using procedures well known in the art

It is also within the scope of the present invention that the immungenic determinant in fact is a mixture of more than one immunogenic determinants as described herein above.

### Vaccine

The vaccines of the present invention comprising collectins are preferably capable of raising a protective immune response in a subject. Such a protective immune response could protect said subject against a variety of clinical conditions, preferably infections by viruses or infections by bacteria.

Such clinical conditions can be selected from the group consisting of Actinomycosis, Adenovirus-infections, Antrax, Bacterial dysentery, Botulisme, Brucellosis (Bang's disease), preferably caused by B. melitensis and B. suis, Candidiasis, Cellulitis, Chancroid, Cholera, Coccidioidomycosis, Acute afebril, Conjunctivitis, Cystitis, Dermatophytosis, Differi, Bacterial Endocarditis, Epiglottitis, Erysipelas, Erysipeloid, Gastroenteritis, Genital herpes, Glandulae, Gonorrhea, Viral Hepatitis, Histoplasmose, Impetigo, Mononucleosis, Influenza, Legionaires disease, Leprosy, Leptospirosis, Lyme disease, Measles, Melioidosis, Meningococcal infection, Meningitis, mumps, Nocardiosis Nocardia asteroides, Non-gonococcal urethritis, Pinta, Pest, Pneumococcal pneumonia, Poliomyelitis, Primary lung infection, Pseudomembraneous enterocolitis, antibiotic-associated Puerperal sepsis, Rabies, Relaps-fever, Rheumatic fever, Rocky Mountain spotted-fever, Rubella, Rubeola, Salmonella infection, Staphylococcal scalded skin syndrome, Steptococcal pneunomia, Streptococcal pharyngitis (strep throat), Syfilis, Tetanus, Toxic shock syndrome, Toxoplasmosis, Tuberculosis, Tularemia, Typhoid fever, Tyfus, Urticaria, Vaginitis, Varicella, Verrucae, Pertussis, Framboesia (Yaws) and Yellow fever.

### Pharmaceutical Composition

The compositions and/or kit of parts of the present invention preferably comprise a pharmaceutical effective amount of at least one collectin and/or collectin homologues as an adjuvant. A pharmaceutical effective amount is an amount of collectin and/or collectin homologues, which in combinantion with an immunogenic determinant is sufficient to induce the desired protective immune response in an individual.

The amount of MBL, which should be administered, depends on the individual, in particular on the size of said individual. Preferably, in the range from 1 ng MBL per kg bodyweight to 1 μg MBL per kg bodyweight is administered per unit dose, more preferably from 5 ng to 500 ng MBL per kg bodyweight is administered per unit dose, even more preferably from 7 ng to 250 ng MBL per kg bodyweight is administered per unit dose, most preferably from 10 ng to 100 ng MBL per kg bodyweight is administered per unit dose.

In one preferred embodiment of the present invention, MBL is administered to an individual in an amount, which does not significantly alter the serum MBL level of said invidual. For example MBL may be administered to an individual, which has reduced serum level of MBL, in an amount which is too small to reconstitute normal MBL serum levels.

The composition or kit of parts according to the present invention, preferably comprise a pharmaceutically effective amount of at least one immunogenic determinant. A pharmaceutically effective amount, is an amount of the immunogenic determinant, which is combination with relevant adjuvants is sufficient to induce the desired protective immune response in an individual.

The amount of immunogenic determinant, which should be administered depends on the individual, in particular on the size of said individual as well as on the immunogenic determinant. Preferably, in the range of 0.01-5 microgram/kg bodyweight of the immunogenic determinant is administered per unit dose, more preferably 0.05 to 4 μg/kg bodyweight, even more preferably from 0.1 to 3 μg/kg bodyweight, most preferably from 0.5 to 2.5 μg/kg bodyweight.

In one embodiment of the present invention the composition comprise one or more additional adjuvants. Such adjuvants may be any compound comprising an adjuvant effect known to the person skilled in the art. For example such adjuvants could be of mineral, bacterial, plant, synthetic or host origin or they could be oil in water emulsions.

Adjuvants could be selected from the group consisting of: AlK(SO₄)₂, AlNa(SO₄)₂, AlNH₄ (SO₄), silica, alum, Al(OH)₃, Ca₃ (PO₄)₂, kaolin, carbon, aluminum hydroxide, muramyl dipeptides, N-acetyl-muramyl-L-threonyl-D-isoglutamine (thr-DMP), N-acetyl-nornuramyl-L-alanyl-D-isoglutamine (CGP 11687, also referred to as nor-MDP), N-acetylmuramyul-L-alanyl-D-isoglutaminyl-L-alanine-2-(1′2′-dipalmitoyl-sn-glycero-3-hydroxphosphoryloxy)-ethylamine (CGP 19835A, also referred to as MTPPE), RIBI (MPL+TDM+CWS) in a 2% squalene/Tween-80.RTM. emulsion, lipopolysaccharides and its various derivatives, including lipid A, Freund's Complete Adjuvant (FCA), Freund's Incomplete Adjuvants, Merck Adjuvant 65, polynucleotides (for example, poly IC and poly AU acids), wax D from Mycobacterium, tuberculosis, substances found in Corynebacterium parvum, Bordetella pertussis, and members of the genus Brucella, liposomes or other lipid emulsions, Titermax, ISCOMS, Quil A, ALUN (see U.S. Pat. Nos. 58,767 and 5,554,372), Lipid A derivatives, choleratoxin derivatives, HSP derivatives, LPS derivatives, synthetic peptide matrixes or GMDP, Interleukin 1 and Interleukin 2.

In one embodiment of the present invention the composition further comprises a carrier. The carrier may be present independently of an adjuvant. The function of a carrier can for example be to increase the molecular weight of the immunogenic determinant in order to increase the activity or immunogenicity of the immunogenic determinant, to confer stability to the determinant, to increase the biological activity of the determinant, or to increase its serum half-life. The carrier may be any suitable carrier known to the person skilled in the art, for example a protein. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier must be a physiologically acceptable carrier acceptable to humans and safe. However, tetanus toxoid and/or diptheria toxoid are suitable carriers in one embodiment of the invention. Alternatively, the carrier may be dextrans for example sepharose.

The immunogenic determinant as well as the collectin and/or collectin homologues of the present invention may be formulated into the vaccine as neutral or salt forms. Pharmaceutically acceptable salts such as acid addition salts, which are formed with free amino groups of peptides and which are formed with inorganic acids such as hydrochloric or phosphoric acids, or such organic acids such as acetic, oxalic, tartaric, maleic, and others known to those skilled in the art. Salts formed with the free carboxyl groups also may be derived from inorganic bases such as sodium, potassium, ammonium, calcium or ferric hydroxides and the like, and such organic bases such as isopropylamine, trimethylamine, 2-ethylamino ethanol, histidine procaine, and others known to those skilled in the art.

The composition of the present invention may be a pharmaceutical composition suitable for parenteral administration. Such compositions preferably, include aqueous and non-aqueous sterile injection solutions which may contain wetting or emulsifying reagents, anti-oxidants, pH buffering agents, bacteriostatic compounds and solutes which render the formulation isotonic with the body fluid, preferably the blood, of the individual; and aqueous and non-aqueous sterile suspensions which may include suspending agents or thickening agents. The pharmaceutical composition may be presented in unit-dose or multi-dose containers, for example, sealed ampoules and vials and may be stored in a freeze-dried condition requiring only the addition of the sterile liquid carrier immediately prior to use.

Preferably, the composition of the present invention comprise one or more suitable pharmaceutical excipients which could be non-sterile or sterile, for use with cells, tissues or organisms, such as a pharmaceutical excipients suitable for administration to an individual. Such excipients may include, but are not limited to, saline, buffered saline, dextrose, water, glycerol, ethanol and combinations of these excipients in various amounts. In one embodiment the diluent is Freunds complete adjuvant. The formulation should suit the mode of administration. The invention further relates to pharmaceutical kit of parts comprising one or more containers filled with one or more of the ingredients of the aforementioned compositions of the invention. Examples of non-aqueous excipients are propylene glycol, polyethylene glycol, vegetable oils such as olive oil, and injectable organic esters such as ethyl oleate.

Preferably, the pharmaceutical compositions of the present invention are prepared in a form which is injectable, either as liquid solutions or suspensions; furthermore solid forms suitable for solution in or suspension in liquid prior to injection are also within the scope of the present invention. The preparation may be emulsified or the immunogenic determinant as well as the collectins and/or collectin homologues according to the present invention may be encapsulated in liposomes.

### Method of Administration

The composition or the kit of parts of the present invention comprising an immunogenic determinant and a collectin and/or collectin homologue can be administered to an individual in need thereof in a manner and in an amount suitable for inducing a desirable immune response in said individual, known to the person skilled in the art.

The immunogenic determinant and the collectin and/or collectin homologue may be administered simultaneously, either as separate formulations or combined in a unit dosage form or they may be administered sequentially. Preferably, the immunogenic determinant and the collectin and/or collectin homologue are administered simultaneously, more preferably, the immunogenic determinant and the collectin and/or collectin homologue are combined in a unit dosage form.

The composition or the kit of parts according to the present invention can be administered alone or in combination with other compounds, such as therapeutic compounds.

Administration could for example be parenteral injection, rapid infusion, nasopharyngeal absorption, dermal absorption, and enterally, such as oral administration. Parenteral injection could for example be intravenous, intramuscular, intradermal or subcutanous. Preferably, said administration is parenterally by injection, more preferably intramuscular or subcutanous injection, most preferably subcutaneous injection at a peripheral anatomical site such as, for human beings, for example, the arm or buttocks or leg.

The number of administrations of the composition or kit of parts according to the present invention as well as the spacing between individual administrations will largely depend on the nature of the immunogenic determininant.

However, the composition or kit of parts according to the present invention are preferably administered to the individual in need thereof at least one times, such as at least two times, for example at least 3 times, such as around 4 times, for example around 5 times. Preferably, such administration is around 3 times.

Preferably, there is at least 1 day between 2 administrations, such as at least 2 days, for example at least 3 days, such as at least 5 days, for example at least one week, such as at least 2 weeks, for example at least one month, such as at least 6 months, for example at least 1 year, such at least 2 years, for example at least 3 years, such as at least 5 years, for example at least 10 years.

In one embodiment of the present invention the collectin(s) and immunogenic determinant(s) according to the present invention are preincubated with other complement factors prior to administration. Said other complement factors could for example be C2, C3, C4 or C5.

Individuals in need of such administration include any animal, preferably any mammal, including a human being, which is susceptible to infections, preferably, infections by viruses or infections by bacteria or infection by fungi or infection by parasites. More preferably, infection that can course one or more of the clinical conditions mentioned herein above.

In one embodiment of the present invention, in which the composition comprise a collectin selected from the group consisting of MBL and MBL homologues, the individual in need of such administration is preferably an individual with a reduced level of MBL in serum, more preferably an individual deficient of MBL. Reduced serum level of MBL is for example less than 500 ng/ml, such as less than 400 ng/ml, for example less than 300 ng/ml, such as less than 200 ng/ml, for example less than 100 ng/ml, such as less than 50 ng/ml. However, any individual susceptible to infections could be an individual in need of such administration.

### EXAMPLES

### Example 1

The example shows that individuals with low serum levels of MBL, have a lower level of mature antibodies compared to normal control individuals.

### Materials and Methods

Serum samples. Sera were available from 20 adults (>16 years of age) with homozygous C2 deficiency, and from 20 healthy blood donors. The selective absence of C2 in the sera was established by rocket electrophoresis, and most of the cases were further characterized on a molecular genetic basis³⁹. All sera were stored in aliquots at −80° C. An additional control group (n=20) consisted of healthy persons with low MBL concentrations¹⁹ and normal C2.

Bacterial antigens. Salmonella typhimurium (serogroup B:O-4,12) SH4809 octasaccharide-polyacrylamide conjugate, Salmonella thompson (serogroup C:O-6,7) IS40 decasaccharide-polyacrylamide conjugate and Salmonella enteritidis (serogroup D:O-9,12) SH 1262 octasaccharide-polyacrylamid conjugate were prepared as described by Chernyak et al.⁴⁰. The conjugates are referred to as BO, CO and DO.

Buffers. Coating buffer: 50 mM carbonate buffer, pH 9.5 for Salmonella oligosaccharides; phosphate-buffered saline, pH 7.2 (PBS) for pneumococcal polysaccharides and monoclonal antibodies. Washing buffers: PBS containing 0.05% Tween (PBS-T) or Tris-buffered saline, pH 7.4 (TBS) containing 0.05% Tween (TBS-T). Diluting buffer in MBL assay: TBS-T with ethylendiamine tetraacetic add (EDTA) at 5 mmol/L. Blocking and diluting buffer: PBS containing 1% bovine serum albumin (PBS-BSA).

Immunoglobulin reagents. Goat anti-human IgG alkaline phosphatase conjugate (γ-chain specific F(ab′)₂ fragment, product no. A-3312, Sigma Biosciences, St. Louis, Mo., U.S.A.); Goat anti-human IgM alkaline phosphatase conjugate (μ-chain specific F(ab′)₂ fragment, product no. A-1067, Sigma); Goat anti-human IgA alkaline phosphatase conjugate (α-chain specific F(ab′)₂ fragment, product no. A-3062, Sigma); Goat anti-human Ig (Fab specific F(ab′)₂ fragment, product no. I-3266, Sigma); Human Serum Protein Calibrator Code No. X 0908 (DAKO, Glostrup, Denmark); Mouse monoclonal anti-MBL (clone 131-1, IgG1, κ. Statens Serum Institut, Copenhagen, Denmark); Control mouse IgG1, κ, M 7894, Sigma).

Reference sera for antibodies to Salmonella BO, CO and DO. To facilitate evaluation of results, antibody concentrations against the Salmonella antigens were expressed in mg/L according to the principle used by Guttormsen et al.⁴¹. Thus, the amount of antigen-fixed antibodies in the enzyme-linked immunosorbent assay (ELISA) system was determined by comparison with results obtained using a human serum protein calibrator in which immunoglobulins were fixed to the solid phase by Fab-specific antibodies. Control experiments were performed with IgA1 and IgA2 κ and λ myeloma proteins of known concentration and with the calibrator to ensure that the Fab-specific antibody bound with comparable efficiency to different immunoglobulins and that there was no significant competition for anti-Fab binding sites at the serum dilutions used.

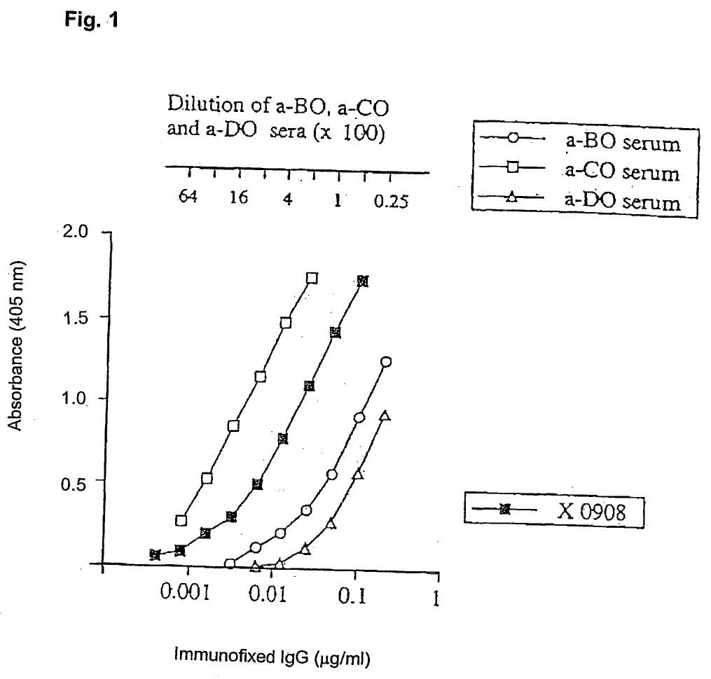

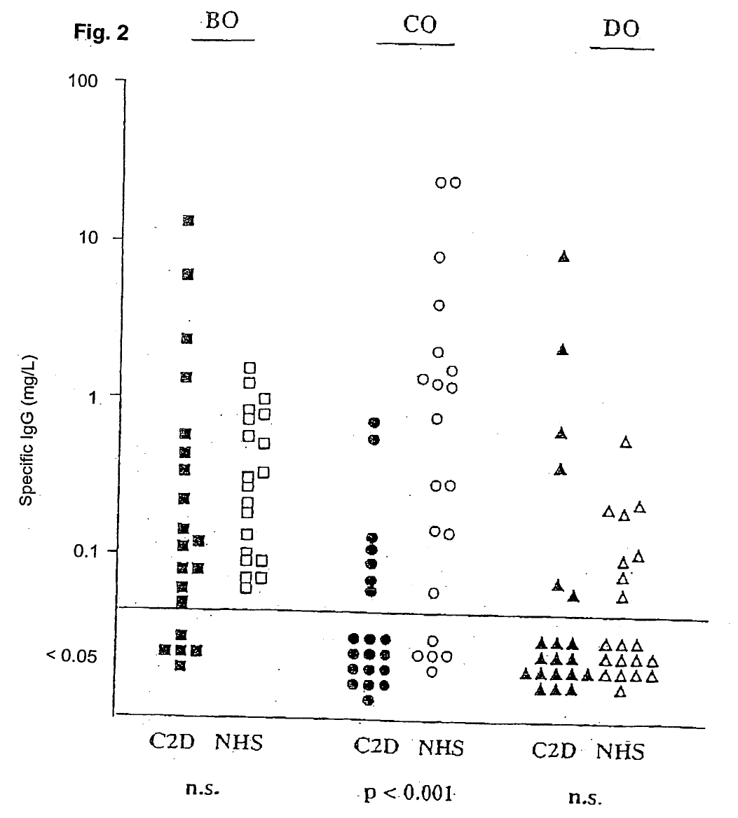

Concentrations of specific IgM and IgG antibodies to Salmonella BO, CO, and DO were assigned to selected sera for subsequent use as operational reference sera. For calibration, one part of a microtiter plate (Nunc-Immunoplates, Maxisorp, A/S Nunc, Kamstrup, Denmark) was coated with Fab specific goat F(ab′)₂ (0.05 ml, 1000 ng/well), while the other part of the plate was coated with Salmonella BO, CO or DO antigen (0.05 ml, 100 ng/well) overnight at 4° C. Three wells were used for for each serum sample, two coated and one uncoated. After washing, the wells were blocked for two hours with PBS-BSA and were then washed again. The human serum protein calibrator was added at appropriate dilutions to the part of the plate coated with Fab specific antibodies. The selected sera containing antibodies to the Salmonella antigens were added in a series of dilutions to the wells coated with Salmonella BO, CO or DO (FIG. 1). After incubation at room temperature for two hours and subsequent washing, alkaline phosphatase-conjugated goat anti-human IgM (1:1000) or IgG (1:10,000) was added. The plate was incubated for two hours at room temperature. The color reaction was developed with p-nitrophenylphosphate (1 mg/ml) in diethanolamine, pH 9.8, for 60 minutes at room temperature. Absorbance was measured at 405 nm in a Multiskan Plus photometer (Labsystems Ltd, Helsinki, Finland). Values obtained were mean absorbance values from coated wells with subtraction of the background absorbance in the uncoated well. Absorbance values for the human serum protein calibrator were plotted against the IgG concentration for each dilution, while absorbance values for specific antibodies were plotted against the serum dilutions. Parallel sections of the two absorbance curves were used for calculation of specific antibody concentrations in the operational reference sera (FIG. 1). The same calibration procedure was used for IgM and IgA.

Antibodies to Salmonella BO, CO and DO. IgM and IgG antibodies to Salmonella BO, CO and DO were determined with ELISA according to the procedure described above. Serum samples were analysed at a dilution of 1:100 or more depending on the antibody concentration. Calibration curves with operational reference sera were included with each plate. Antibody concentrations are given in mg/L.

MBL concentrations. MBL was measured with ELISA. The general procedure has been described previously⁴² and was based on the use of monoclonal anti-MBL (clone 131-1) as a capture antibody and as a biotinylated second antibody. Another mouse IgG1 kappa protein was used for coating of background control wells. MBL in the group with low MBL was was also estimated by time resolved immuno fluorometric assay (TRIFMA)¹⁹. In brief, dilutions of plasma or serum were added to microtitre plate wells coated with monoclonal anti-MBL (clone 131-1). After incubation and wash, development was carried out with secondary europium-labelled monoclonal anti-MBL. Bound europium was estimated on a time resolved fluorometer. A calibrator plasma was calibrated against a purified MBL preparation, which had been subjected to quantitative amino acid analysis. The same calibrator was used for ELISA and TRIFMA determinations.

Statistics. Statistical analysis was performed with the two-tailed Mann-Whitney test and the Spearman rank correlation test.

### Results and Discussion

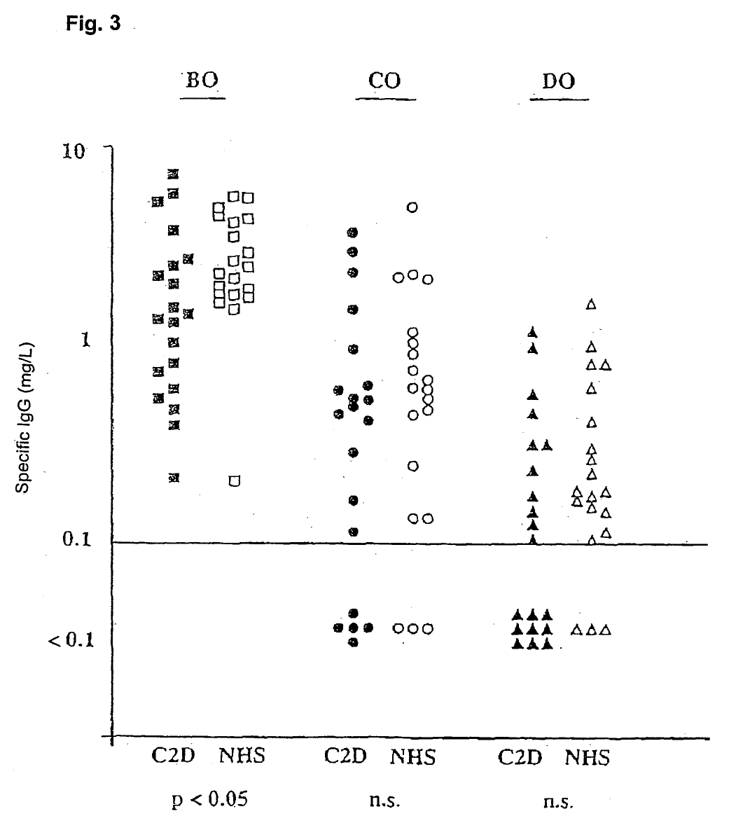

The central finding of the study was the marked difference between the C2 deficient group and the control group with regard to concentrations of IgG antibodies to the Salmonella CO antigen (p<0.001). The median values were <0.05 mg/L and 0.54 mg/L, respectively (FIG. 2). The control group showed higher concentrations of IgM antibodies to BO than the C2 deficient group (p<0.05), and a similar tendency was noted for IgM against CO and DO (FIGS. 2 and 3). We suggest that the discrepancy between the IgM and the IgG responses to CO in the C2 deficient group was caused by impaired isotype switching.

Isotype switching from IgM to IgG of different subclasses occurs in the order IgG3 and IgG1 followed by IgG2 and IgG4 in accordance with the organization of the heavy chain locus⁴³. The gradual increase of isotype concentrations during ontogeny with IgM, IgG3 and IgG1 reaching adult levels early in life, in contrast to the slow development of IgG2 and IgG4^{44,45}, indicates that the switching process is operative during immune system maturation. In hereditary C3 deficiency and in deficiencies of C1, C2 or C4, serum concentrations of the IgG2 and IgG4 subclasses have been shown to be lower than normal, suggesting that C3 and a functional classical pathway promote this process⁴⁶. With regard to specific antibodies, C3-deficient patients had low levels of IgG against mixed capsular polysaccharides of S. pneumoniae⁴⁷. By contrast, specific IgG levels in patients with C1, C2 or C4 deficiencies were largely normal.

The present finding for the Salmonella CO response provides the first clear suggestion of MBL pathway C3 convertase-dependent impairment of IgM/IgG switching for specific antibodies during immune system maturation.

Salmonella CO differs from BO and DO by its reactivity with MBL³⁸. MBL recognizes carbohydrate structures such as N-acetylglucosamine and mannose, and is thought to recruit C3 through activation of the classical pathway C3 convertase, C4bC2a, by the MBL/MASP1/MASP2 complex⁷. In our study, the implication that the poor IgG response to CO in the C2 deficient group might involve a lectin pathway-dependent mechanism prompted analysis of MBL in the sera, and supplementary investigation of antibody responses to CO in a group with low MBL levels.

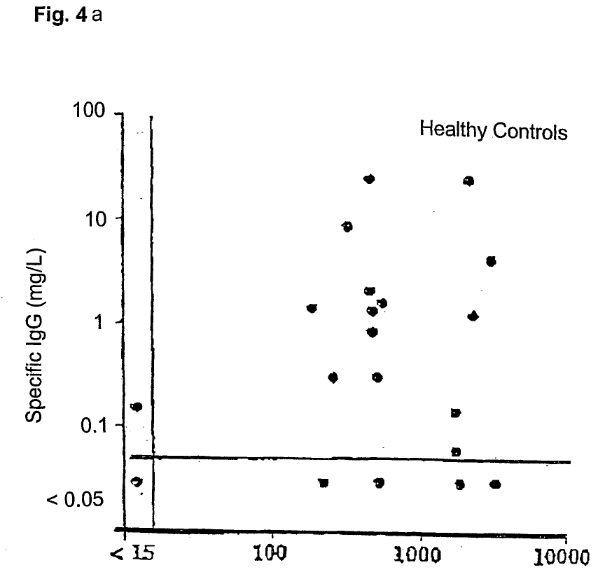

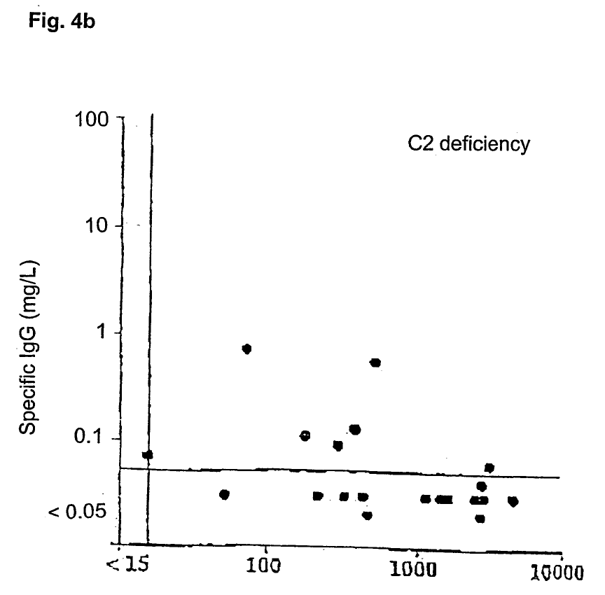

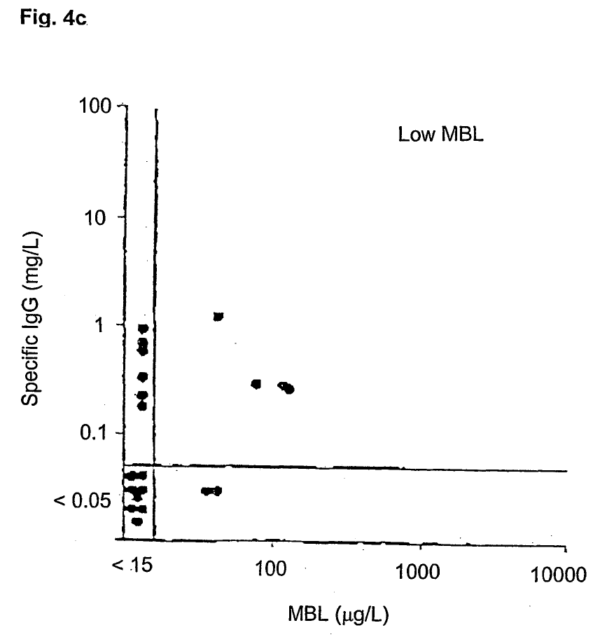

MBL was measured with ELISA in the three categories (FIG. 4). The healthy controls and the C2-deficient persons showed MBL concentrations within the same broad range. The group with low MBL had values in the range between 6 and 80 mg/L as determined by TRIFMA and values between <15 and 130 mg/L as determined with ELISA. A moderate discrepancy of results between the methods was noted for two sera.

When MBL levels were compared with specific IgG against CO it was evident that the decreased responses in the C2-deficient group were not related to low MBL concentrations (FIG. 4, top and middle). Two members of the unselected control group had very low MBL (<15 mg/L ) and also showed poor IgG responses to CO. The critical finding was that concentrations of specific IgG against CO were consistently low with a median of 0.11 mg/L in the group with low MBL (FIG. 4, bottom). For statistical analysis, the two unselected controls with low MBL were included in the group with low MBL. Compared with healthy MBL-sufficient controls (n=18), the persons with low MBL (n=22) had significantly lower (p<0.01) concentrations of specific IgG against CO. Like the C2-deficient group, the group with low MBL had normal concentrations of specific IgM against the CO antigen.

Lutz⁴⁹ recently discussed the basis of primary immune responses to nonself, and suggested that formation of complement-activating immune complexes containing antigen and natural antibody may play a critical role in vivo. This concept could be valid for thymus-dependent as well as for some thymus-independent antigens. In the present study, IgM antibodies against BO were found to be moderately low in the C2 deficient group (FIG. 3), but otherwise the findings were not suggestive of a role for the classical pathway in triggering of IgM responses.

The question emerges if reduced immune responses, secondary to complement dysfunction, contributes to the decreased immunity encountered in conditions associated with impaired recruitment of C3^{22,23}. An important point is that immune responses to thymus-independent antigens mainly appears to require complement when antigen is presented at low concentrations^{24,34,50}.

Maturation of carbohydrate immunity develops slowly during ontogeny⁵¹ and might partly be determined by a course of subclinical immunisation events with exposure to low doses of antigen. In this way, it is conceivable that complement deficiency might delay establishment of immunity. On the other hand, distinct vaccination responses to polysaccharides have been documented in deficiencies of C3^{35,36} and properdin^{36,52}, a component of the alternative activation pathway, and might also be expected in classical pathway deficiency states.

The present findings point to impaired immune responses against some carbohydrate antigens in C2 deficiency. It is suggested that the MBL pathway could be critically involved in isotype switching during immune system maturation.

The impact of complement dysfunction on immune responsiveness appears to reflect interesting links between innate and acquired immunity and has clinical significance with regard to establishment of immunity in inherited and acquired complement deficiency states.

### Example 2

Transgenic mice wherein one of the two MBL genes, MBL-A and MBL-C, are silenced through homologous recombination are created. These mice are bred and through cross breeding double MBL deficient mice are created.

Mice are divided into groups:

MBL deficient

MBL sufficient (wild type)

MBL deficient mice reconstituted with MBL.

Each group of mice are immunised with the following antigens:

1) polysaccharides (mannan purified from yeast)

2) polysaccharide-based pneumococcal vaccine, (Pneumovax II);

3) glycoproteins (ovalbumin and/or HbsAg)

4) ovalbumin and/or HbsAg, wherein the carbohydrate has been removed by treatment with glycosidases

5) neo glycoproteins (serum albumin, which have been glycosylated by chemical coupling of sugars)

6) whole encapsulated virus expressing glycosylated envelope proteins (influenza A virus)

7) Influenza A virus grown at conditions inhibiting the glycosylation through the addition of tunicamycin to the culture medium.

Each antigen is given in doses ranging from 10 ng to 10 μg per dose. Furthermore, each antigen is administered both with and without premixing with MBL. The MBL administered is purified, recombinant, human MBL, produced as described in international patent application 00/70043. Different doses of MBL are administered varying from the same amount as the antigen to ten fold excess.

The antigen and/or MBL dose is given subcutaneously, both with and without preadsorption to alluminum hydroxide as an unspecific adjuvans. Each animal is immunised twice with the same antigen with one month between the first and the second immunisation. The mice are bled two weeks after the first immunisation and one week after the second immunisation and the serum prepared for antibody assessment.

The antibody responses are evaluated by ELISA. Wells of 96 well microtiter plate are first coated with the antigen, then dilutions of the murine serum from 1/100 to 1/1,000,000 is added, and finally, after washing, bound mouse antibody is quantified by adding alkaline-phosphatase-labelled rabbit anti-mouse Ig (H and L chain). The antibody titre, i.e., the dilution capable of 30% of maximal binding is determined and compared between the different groups.

### REFERENCES

1) Law, S. K. A. & Reid, K. B. M. Complement, 2. ed. (Ed. Male, D.) 1-88 (In Focus, IRL Press, Oxford, 1996).

2) Ikeda, K., Sannoh, T., Kawasaki, N., Kawasaki, T. & Yamashina, I. Serum lectin with known structure activates complement through the classical pathway. J. Biol. Chem. 262, 7451-7454 (1987).

3) Kawasaki, T., Etoh, R. & Yamashina, I. Isolation and characterization of a mannan-binding protein from rabbit liver. Biochem. Biophys. Res. Commun. 81, 1018-1024 (1978).

4) Matsushita, M. & Fujita, T. 4) Activation of the classical complement pathway by mannose-binding protein in association with a novel C1s-like serine protease J. Exp. Med. 176, 1497-1502 (1992).

5) Ji, Y-H. et al. Activation of the C4 and C2 components of complement by a proteinase in serum bactericidal factor, Ra reactive factor J. Immunol. 150, 571-578 (1993).

6) Thiel S, Vorup-Jensen T, Stover C M, Schwaeble W, Laursen S B, Poulsen K, Willis A C, Eggleton P, Hansen S, Holmskov U, Reid K B and Jensenius J C (1997) A second serine protease associated with mannan-binding lectin that activates complement. Nature, 386(6624): 506-510

7) Turner, M. W. Mannose-binding lectin: the pluripotent molecule of the innate immune system. Immunol. Today, 17, 532-540 (1996).

8) Kawasaki, N., Kawasaki, T. & Yamashina, I. A serum lectin (mannan-binding protein) has complement-dependent bactericidal activity. J. Biochem. 106, 483-489 (1989).

9) Kuhlman, M., Joiner, K. & Ezekowitz, R. A. B. The human mannose-binding protein functions as an opsonin. J. Exp. Med. 169, 1733-1745 (1989).

10) Sumiya, M. et al. Molecular basis of opsonic defect in immunodeficient children. Lancet 337, 1569-1570 (1991).

11) Lipscombe, R. J. et al. High frequencies in African and non-African populations of independent mutations in the mannose binding protein gene. Hum. Mol. Genet. 1, 709-715 (1992).

12) Madsen H. O. et al. A new frequent allele is the missing link in the structural polymorphism of the human mannan-binding protein. Immunogenetics 40, 37-44 (1994).

13) Super, M., Thiel, S., Lu, J., Levinsky, R. J. & Turner, M. W. Association of low levels of mannan-binding protein with a common defect of opsonisation. Lancet ii, 1236-1239 (1989).

14) Garred, P., Madsen, H. O., Hofmann, B. & Svejgaard, A. Increased frequency of homozygosity of abnormal mannan-binding-protein alleles in patients with suspected immunodeficiency. Lancet 346, 941-943 (1995).

15) Summerfield J A, Sumiya M, Levin M and Turner M W (1997) Association of mutations in mannose-binding protein gene with childhood infection in consecutive hospital series. BioMed J 314: 1229-1232

16) Summerfield, J. A. et al. Mannose binding protein gene mutations associated with unusual and severe infections in adults. Lancet 345, 886-889 (1995).

17) Nielsen, S. L., Andersen, P. L., Koch, C., Jensenius, J. C. & Thiel, S. The level of the serum opsonin, mannan-binding protein in HIV-1 antibody-positive patients. Clin. Exp. Immunol. 100, 219-222 (1995).

18) Garred, P., Madsen, H. O., Balslev, U., Hofmann, B., Pedersen, C., Gerstoft, J. and Svejgaard, A. Susceptibility to HIV infection and progression of AIDS in relation to variant alleles of mannose-binding lectin. Lancet 349, 236-240 (1997).

19) Christiansen, O. B., Kilpatrick, D. C., Souter, V., Varming, K., Thiel, S., Jensenius, J. C. (1999) Mannan-binding lectin deficiency is associated with unexplained recurrent miscarriage. Scand. J. Immunol., 49, 193-196

20) Davies, E. J., Snowden, N., Hillarby, M. C., Carthy, D. Grennan, D. M., Thomson, W. and Ollier, W. E. R. Mannose-binding protein gene polymorphism in systemic lupus erythematosus. Arthritis Rheum. 38, 110-114 (1995).

21) Garred P, Pressler T, Madsen H O, Frederiksen B, Svejgaard A, Hoiby N, Schwartz M and Koch C. Association of mannose-binding lectin gene heterogeneity with severity of lung disease and survival incystic fibrosis. J Clin Invest. 104, 431-7 (1999).

22) Valdimarsson H, Stefansson M, Vikingsdottir T, Arason G J, Koch C, Thiel S and Jensenius J C (1998) Reconstitution of opsonizing activity by infusion of mannan-binding lectin (MBL) to

23) Dempsey P W, Allison M E D, Akkaraju S, Goodnow C C, Fearon D T. C3d of complement as a molecular adjuvant: Bridging innate and acquired immunity. Science 1996; 271:348-50.

24) Pepys M B. Role of complement in induction of antibody production in vivo. J Exp Med 1974; 140:126-145.

25) Jackson C G, Ochs H D, Wedgewood R J. Immune response of a patient with deficiency of the fourth componenet of complement and systemic lupus erythematosus. N Engl J Med 1979; 300:1124-9.

26) Bitter-Suermann D, Burger R. Guinea pigs deficient in C2, C4, C3 or the C3a receptor. Progr Allergy 1986; 39:134-58.

27) O'Neil K M, Ochs H D, Heller S R, Cork L C, Morris J M, Winkelstein J A. Role of C3 in humoral immunity. Defective antibody production in C3-deficient dogs. J Immunol 1988; 140:1939-45.

28) Ochs H D, Nonoyama S, Zhu Q, Farrington M, Wedgwood R J. Regulation of antibody responses: The role of complement and adhesion molecules. Clin Immunol Immunopathol 1993; 67:33-40.

29) Kinoshita T. Complement receptors and regulation of humoral immune response. Cruse J M, Lewis R E Jr (eds) Complement Today 1993; 1: 46-55.

30) Molina H, Holers V M, Li B, Fang Y F, Mariathasan S, Goellner J, Strauss-Schoenberger J, Karr R W, Chaplin D D. Markedly impaired humoral immune response in mice deficient in complement receptors 1 and 2. Proc Nat Acad Sci (USA) 1996; 93:3357-61.

31) Croix D A, Ahearn J M, Rosengard A M, Han S, Kelsoe G, Ma M, Carroll M C. Antibody response to a T-dependent antigen requires B cell expression of complement receptors. J Exp Med 1996; 183:1857-64.

32) Sjöholm A G. Inherited complement deficiency states: implications for immunity and immunological disease. APMIS 1990; 98:61-874.

33) Figueroa J E, Densen P. Infectious diseases associated with complement deficiencies. Clin Microbiol Rev 1991; 4:359-95.

34) Matsuda T, Martinelli G P, Osler A G. Studies of immunosuppression by cobra venom factor II. On responses to DNP-Ficoll and DNP-polyacrylamide. J Immunol 1978; 121:2048-51.

35) Alper C A, Colten H R, Gear J S S, Rabson A R, Rosen F S. Homozygous human C3 deficiency. The role of C3 in antibody production, C1s-induced vasopermeability, and cobra venom-induced passive hemolysis. J Clin Invest 1976; 57: 222-9.

36) Fijen C A P, Kuijper E J, Drogari-Apiranthitou M, van Leeuwen Y, Daha M R, Dankert J. Protection against meningococcal serogroup ACYW disease in complement-deficient individuals vaccinated with the tetravalent meningococcal capsular polysaccharide vaccine. Clin Exp Immunol 1998; 114:362-9.

37) Grossman N, Svensson B, Leive L, Lindberg M. Salmonella O antigen-specific oligosaccharide-octyl conjugates activate complement via the alternative pathway at different rates depending on the structure of the O antigen. Molecular Immunol 1990; 27: 859-65.

38) Schweinle J E, Ezekowitz R A B, Tenner A J, Kuhlman M, Joiner K A. Human mannose-binding protein activates the alternative complement pathway and enhances serum bactericidal activity on a mannose-rich isolate of Salmonella. J Clin Invest 1989; 84: 1821-9.

39. Truedsson L, Alper C A, Awdeh Z L, Johansen P, Sjöholm A G, Sturfelt G. Characterization of type I complement C2 deficiency MHC haplotypes. Strong conservation of the complotype/HLA-B-region and absence of disease association due to linked class II genes. J Immunol 1993; 151:5856-63.

40. Chernyak A, Weintraub A, Norberg T, Kallin E. Preparation of oligosaccharide-polyacrylamide conjugates and their use as antigens in enzyme immunoassey (EIA). Glycoconjugate J 1990; 7: 111-120.

41. Guttormsen H-K, Wetzler L M, Naess A. Humoral immune response to the class 3 outer membrane protein during the course of meningococcal disease. Infect Immun 1993; 61:4734-42.

42. Garred P, Madsen H O, Kurtzhals J A L et al. Diallelic polymorphism may explain variations of blood concentration of mannan-binding protein in Eskimoes but not in black Africans. Eur J Immunogenet 1992; 19:403-12.

43. Rathbun G, Berman J, Yancopoulos G, Alt F W. Organization and expression of the mammalian heavy-chain variable-region locus. In: Honjo T, Alt F W, Rabbitts T H, eds. Immunoglobulin Genes. London: Academic Press, 1989:63-90.

44. Johansson S G O, Berg T. Immunoglobulin levels in healthy children. Acta Paediatr Scand 1967; 56:572-9.

45. Oxelius V-A. Quantitative and qualitative investigations of serum IgG subclasses in immunodeficiency diseases. Clin Exp Immunol 1979; 36:112-6.

46. Bird P, Lachmann P J. The regulation of IgG subclass production in man: low serum IgG4 in inherited deficiencies of the classical pathway of C3 activation. Eur J Immunol 1988; 18: 1217-22.

47. Hazlewood M A, Kumaratne D S, Webster A D B, Goodall M, Bird P, Daha M. An association between homozygous C3 deficiency and low levels of anti-pneumococcal capsular polysaccharide antibodies. Clin Exp Immunol 1992; 87: 404-9.

48. Fearon D T, Locksley R M. The instructive role of innate immunity in the acquired immune response. Science 1996; 272: 50-4.

49. Lutz H U. How preexisting, germline-derived antibodies and complement may help induce a primary immune response to nonself. Scand J Immunol 1999; 49: 224-8.

50. Fehr T, Bachmann M F, Bluethmann H, Kikutani H, Hengartner H, Zinkernagel R M. T-independent activation of B cells by vesicular stomatitis virus: no evidence for the need of a second signal. Cell Immunol 1996; 168:184-92.

51. Stein K E. Thymus-independent and thymus-dependent responses to polysaccharide antigens. J Infect Dis 1992; 165 (suppl 1):S49-52.

52. Söderström C, Braconier J H, Käyhty H, Sjöholm A G, Thuresson B. Immune response to tetravalent meningococcal vaccine: opsonic and bactericidal functions of normal and properdin deficient sera. Eur J Clin Microbiol Infect Dis 1989; 8:220-4.

1. A vaccine composition comprising an effective amount of at least one collectin and/or collectin homologue and at least one immunogenic determinant.

2. The vaccine composition according to claim 1, wherein said at least one collectin and/or collectin homologue comprise at least one subunit, such as 2 subunits, for example 3 subunits, such as 4 subunits, for example 5 subunits, such as 6 subunits.

3. The vaccine composition according to claim 1, wherein said collectin and/or collectin homologue comprise a mixture of collectins and/or collectin homologues, each comprising a different number of subunits.

4. The vaccine composition according to claim 2, wherein said subunits each comprise 3 individual collectin polypeptide chains.

5. The vaccine composition according to claim 4, wherein the collectin polypeptide chains each comprises an N-terminal region cysteine rich region, a collagen-like domain, a neck region and/or a carbohydrate recognition domain (CRD).

6. The vaccine composition according to claim 1, wherein said at least one collectins are selected from the group consisting of. SP-A, SP-D, CL43, conglutinin, CL1 and mannose bidning lectin (MBL).

7. The vaccine composition according to claim 1, wherein said collectin is MBL and/or a MBL homologue.

8. The vaccine composition according to claim 7, comprising in then range from 1 ng to 1 μg MBL per kg bodyweight of an individual to receive the vaccination and at least one immunogenic determinant for the use as vaccination.

9. The vaccine composition according to claim 7, comprising in then range from 1 ng to 100 ng MBL per kg bodyweight of an individual to receive the vaccination and at least one immunogenic determinant for the use as vaccination.

10. The vaccine composition according to claim 7, wherein said MBL and/or a MBL homologue is human MBL.

11. The vaccine composition according to claim 7, wherein said MBL is naturally occuring MBL.

12. The vaccine composition according to claim 7, where MBL has been purified from mammalian plasma, most preferred human plasma.

13. The vaccine composition according to claim 7, wherein MBL is recombinant and expressed from a gene expression construct comprising nucleotide sequences encoding MBL polypeptides or funtional equivalents thereof operably linked to expression signals not natively associated therewith.

14. The vaccine composition according to claim 13, wherein said nucleotide sequences are cDNA sequences.

15. The vaccine composition according to claim 13, wherein said nucleotide sequences are genomic DNA sequences.

16. The vaccine composition according to any of claims 7, wherein MBL is recombinant and produced in a host selected from the group consisting of: transgenic animal, mammalian cell line, which includes human cell line, insect cells, yeast cells, bacterial cells and plants.

17. The vaccine composition according to claim 7, where MBL is recombinant and at least 50% of the MBL oligomers has an apparent molecular weight higher than 200 kDa, when analysed by SDS-PAGE and/or Western blot.

18. The vaccine composition according to claim 7, wherein MBL is recombinant and at least 95% of the MBL oligomers has an apparent molecular weight higher than 200 kDa, when analysed by SDS-PAGE and/or Western blot.

19. The vaccine composition according to claim 7, wherein said MBL comprise a ratio of tetramers, pentamers and/or hexamers to dimers of MBL subunits of at least 2:1.

20. The vaccine composition according to any of claims 1 and 7, wherein at least one immunogenic determinant is a bacterial antigen.

21. The vaccine composition according to any of claims 1 and 7, wherein at least one immunogenic determinant is a polysaccharide.

22. The vaccine composition according to any of claims 1 and 7, wherein at least one immunogenic determinant is a Salmonella antigen

23. The vaccine composition according to any of claims 1 and 7, wherein at least one immunogenic determinant is a Salmonella O-antigen

24. The vaccine composition according to any of claims 1 and 7, wherein at least one immunogenic determinant is a Steptococcus pneunomiae antigen.

25. The vaccine composition according to any of claims 1 and 7, wherein at least one immunogenic determinant is a meningococcus antigen.

26. The vaccine composition according to any of claims 1 and 7, wherein at least one immunogenic determinant is a hepatitis B virus antigen.

27. The vaccine composition according to any of claims 1 and 7, wherein at least one immunogenic determinant is an influenza virus antigen.

28. The vaccine composition according to any of claims 1 and 7, wherein at least one immunogenic determinant comprise at least one saccharide, selected from the group consisting of mono-saccharides, di-saccharides, tri-saccharides, poly-saccharides.

29. The vaccine composition according to any of claims 1 and 7, wherein at least one immunogenic determinant comprise at least one saccharide, selected from the group consisting of mono-saccharides, di-saccharides, tri-saccharides, poly-saccharides, not natively associated therewith.

30. The vaccine composition according to claim 28, wherein said saccharide comprise mannan.

31. The vaccine composition according to any of claims 1 and 7, wherein at least one immunogenic determinant is conjugated with MBL.

32. A method for immunising an individual against an immunogenic determinant, comprising administering to said individual at least one collectin and/or collectin homologue and at least one immunogenic determinant selected from the group consisting of bacterial, fungal, viral and other pathogenic immunogenic determinants and any derivative of such infectious agents.

33. The method according to claim 32, wherein said collectin is MBL and/or a MBL homologue.

34. The method according to claim 32, wherein said collectin and/or collectin homologue and at least one immunogenic determinant is administered in a form selected from intravenous, intramuscular, subcutanous or oral administration.

35. The method according to claim 32, where collectin and/or collectin homologue and at least one immunogenic determinant is administered simultaneously, either as separate formulations or combined in a unit dosage form.

36. Use of a pharmaceutical acceptable amount of at least one collectin and/or collectin homologue for the preparation of a pharmaceutical composition for improvement of the immune response following vaccination with bacterial, fungal, viral and/or other pathogenic immunogenic determinants and/or any derivative of such infectious agents.

37. The use according to claim 36, wherein said collectin is MBL and/or a MBL homologue.

38. Use of a pharmaceutical acceptable amount of at least one collectin and/or collectin homologue and at least one immunogenic determinant selected from the group consisting of bacterial, fungal, viral and other pathogenic immunogenic determinants and any derivative of such infectious agents for the preparation of a vaccine composition.

39. A kit of parts comprising a pharmaceutically effective amount of a collectin and/or a collectin homologue and at least one immunogenic determinant for use as vaccination.

40. The kit of parts according to claim 39, wherein said collectin is MBL and/or MBL homologue.
